SEQUENCE LISTING (8 sequences)

SEQ ID NO: 1 - DNA, 20 bp, Artificial Sequence
  Description of Artificial Sequence Synthetic primer
agagctacgagctgcctgac

SEQ ID NO: 2 - DNA, 20 bp, Artificial Sequence
  Description of Artificial Sequence Synthetic primer
agcactgtgttggcgtacag

SEQ ID NO: 3 - DNA, 22 bp, Artificial Sequence
  Description of Artificial Sequence Synthetic primer
agatctcggcgaagcaaagagt

SEQ ID NO: 4 - DNA, 24 bp, Artificial Sequence
  Description of Artificial Sequence Synthetic primer
cggcatccagaagttttctcacac

SEQ ID NO: 5 - DNA, 24 bp, Artificial Sequence
  Description of Artificial Sequence Synthetic primer
aggacatgtcggatggcttcatct

SEQ ID NO: 6 - DNA, 22 bp, Artificial Sequence
  Description of Artificial Sequence Synthetic primer
tcttgcagccgaaacagagctt

SEQ ID NO: 7 - DNA, 18 bp, Artificial Sequence
  Description of Artificial Sequence Synthetic primer
ctctcggcctcagactcc

SEQ ID NO: 8 - DNA, 18 bp, Artificial Sequence
  Description of Artificial Sequence Synthetic primer
agggaggaggcgactgca

(19) United States
(12) Patent Application Publication
(10) Pub. No.: US 2019/0234952 A1
(43) Pub. Date: Aug. 1, 2019

(54) METHODS OF DETECTING HYPOXIA-ASSOCIATE PEPTIDES

(71) Applicant: ARIZONA BOARD OF REGENTS ON BEHALF OF ARIZONA STATE UNIVERSITY, Scottsdale, AZ (US)

(72) Inventor: Ye Hu, Scottsdale, AZ (US)

(21) Appl. No.: 16/211,040

(22) Filed: Dec. 5, 2018

Related U.S. Application Data
(60) Provisional application No. 62/594,984, filed on Dec. 5, 2017.

(51) Int. Cl. G01N 33/574; G01N 33/68

(52) U.S. Cl. CPC G01N 33/57438; G01N 33/6848

(57) ABSTRACT

In certain embodiments, the present invention provides a method of detecting hypoxia status in a tumor cell by detecting a level of hydroxyprolyl BK (Hyp-BK) peptides in a patient sample, and detecting a level of bradykinin (BK) peptides the patient sample, wherein a change in the Hyp-BK/BK ratio as compared to the Hyp-BK/BK ratio in a comparable normal cell is a marker for tumor hypoxia.

### PRIORITY

This application claims benefit of priority to U.S. Provisional Application Ser. No. 62/594,984 filed Dec. 5, 2017, which application is incorporated by reference herein.

### BACKGROUND

Tumor hypoxia is a clinically important feature because it is closely linked to poor patient outcomes. Physiological and biochemical adaptations in hypoxic tumors are exemplified by elevated glucose uptake, an acidified tumor microenvironment, and progressively evolved malignancy. Accumulation of hypoxic features promotes the growth of cancer stem cells and increases the risk of metastasis, thereby limiting the effectiveness of surgeries and inducing resistance to chemotherapies and radiotherapies. Effective detection and characterization of tumor hypoxia will therefore provide predictive information regarding personalized treatment plans.

At present, the gold standard for characterizing tumor hypoxia involves the use of polarographic electrodes; however, this method is quite invasive. Less invasive methods, such as electron paramagnetic resonance, 19F-magnetic resonance spectroscopy, Overhauser-enhanced magnetic resonance imaging (MRI), photoacoustic tomography, sonography, and hypoxia positron emission tomography (PET) imaging are difficult to implement in the clinic mainly due to regulatory restraints, safety concerns, and uncertain correlations with prognoses. Moreover, imaging methods do not yield accurate information regarding hypoxic conditions owing to the rapid dynamic changes in hypoxia. Endogenous hypoxic markers, such as hypoxia-inducible factor-1α (HIF-1α), carbonic anhydrase IX, and glucose transporter 1, are inconvenient or impractical for clinical measurement and are associated with poor specificity and sensitivity. In vitro assays, such as the integrated immunohistochemistry (IHC) panel of multiple hypoxic protein markers and comet assays, do not provide information quickly. Thus, there is an urgent need for identification of biomarkers of hypoxia that can be detected rapidly, conveniently, and with accuracy for development of personalizing treatment plans and to improve prediction of therapeutic responses.

### SUMMARY

The invention aims to address this need by combining the advantages of nanotechnology and blood-based peptidomics to develop a quick, sensitive detection platform for monitoring hypoxia-specific conditions from blood samples.

In certain embodiments, the present invention provides a method of detecting hypoxia status in a tumor cell comprising (a) detecting a level of hydroxyprolyl BK (Hyp-BK) peptides in a patient sample, and (b) detecting a level of bradykinin (BK) peptides the patient sample, wherein an increased Hyp-BK/BK ratio as compared to the Hyp-BK/BK ratio in a comparable normal cell is a marker for tumor hypoxia.

In certain embodiments, the present invention provides a method of detecting hypoxia status in a tumor cell comprising (a) detecting a level of hydroxyprolyl BK (Hyp-BK) in a patient sample, and (b) detecting a level of bradykinin (BK) the patient sample, wherein BK and Hyp-BK peptides from the patient sample are subjected to nanopore-based fractionation and analyzed by matrix-assisted laser desorption/ionization time-of-flight mass spectrometry (MALDI-TOF MS).

In certain embodiments, the present invention provides a method of treating a hyperproliferative disorder in a patient in need thereof comprising administering to a patient identified as having a low plasma hydroxyprolyl BK (Hyp-BK)/protein bradykinin (BK) ratio (as compared to a normal or control level in patient) a pharmaceutical composition comprising a chemotherapeutic agent.

In certain embodiments, the present invention provides a method of treating hyperproliferative disorder in a patient in need thereof comprising administering to a patient identified as having a high plasma hydroxyprolyl BK (Hyp-BK)/protein bradykinin (BK) ratio (as compared to a normal or control level in patient) a pharmaceutical composition comprising a chemotherapeutic agent.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1E. P4Hα1 is upregulated through the HIF-1α pathway under hypoxia in pancreatic cancer cell Colo357. (FIG. 1A) HIF-1α, P4Hα1 and VEGF protein levels were elevated after 48 h culture in hypoxic than normoxic conditions. (FIG. 1B) The mRNA levels of HIF-1α and P4Hα1 were upregulated in hypoxic pancreatic cancer cell Colo357 than normoxic cells. (FIG. 1C) Enriched binding of HIF-1α to the P4Hα1 promoter was approximate three times higher in the hypoxic cells Colo357 than the normoxic ones as detected by ChIP-PCR, which confirmed that P4Hα1 is subject to direct regulation by HIF-1α. (FIG. 1D) The P4Hα1 protein has been effectively enriched both in the hypoxic and normoxic tumor cells by specific antibody and protein A beads. (FIG. 1E) Immunofluorescence microscopy showed translocation of HIF-1α into nucleus after hypoxic induction in pancreatic cancer cell line Colo357.

FIGS. 2A-2D. P4Hα1 regulates Hyp-BK production by hypoxic pancreatic cancer cells. (FIG. 2A) Immunoprecipitated BK from Colo357 cells cultured under hypoxic conditions combines higher levels of P4Hα1, compared with normoxic cells. (FIG. 2B) Immunoprecipitated P4Hα1 from Colo357 cells cultured under hypoxic conditions produces significantly higher levels of Hyp-BK/BK, compared with normoxic cells (p=0.0142). When a HIF-1α specific inhibitor, digoxin (200 nM), is included, under the hypoxic conditions, the ratio is significantly reduced (p=0.0131). (FIG. 2 FIG. 2C) Cancer cells appeared to be more responsive to hypoxia as differential change of P4Hα1 and VEGF (i.e., hypoxia vs. normoxia) was more obvious in cancer cells than the normal ones. (FIG. 2D) Pancreatic cancer cells and non-malignant pancreatic cells expressing a K-Ras mutant allele catalyze more Hyp-BK during hypoxia than normal pancreatic cells.

FIGS. 3A-3C. The expression of P4Hα1 is correlated with patient survival. (FIG. 3A) P4Hα1 is highly expressed in tissues of patients with PDAC, compared with chronic inflammation and normal adjacent tissue. (FIG. 3B) The expression level of P4Hα1 expression level is correlated with tumor stage (p<0.05). (FIG. 3C) P4Hα1 expression level is correlated with patient survival (p<0.001).

FIGS. 4A-4D. Hyp-BK/BK indicates tumor hypoxic condition and is inversely correlated with clinical prognosis of anticancer therapy. (FIG. 4A) Examples of P4Hα1 and CA9 staining in human pancreatic cancer. (FIG. 4B) P4Hα1 expression level increases as the hypoxic marker CA9 increases in tissues of patients with PDAC. (FIG. 4C) Measurement of Hyp-BK/BK in serum samples from 40 clinical pancreatic cancer patients has shown that there was a significant correlation between high levels of Hyp-BK/BK and P4Hα1 staining score (p=0.0100). (FIG. 4D) Measurement of Hyp-BK/BK in plasma samples from 34 clinical pancreatic cancer patients has shown that there was a significant correlation between high levels of Hyp-BK/BK and poor treatment response (p=0.0357).

FIG. 5. Hypoxia-induced P4Hα1 promotes Hyp-BK/BK in patients with pancreatic cancer, which could be a marker to reflect the hypoxic status.

FIGS. 6A-6C. (FIG. 6A) P4Hα1 protein level was increased with length of culture under hypoxic conditions. (FIG. 6B) HIF-1α/P4Hα1 cytoplasmic and nuclear protein expression increased in Colo-357 cells after hypoxic treatment. (FIG. 6C) Western blot analysis of lysates from four different pancreatic cell lines (AsPC-1, BxPC-3, MIA, and PANC-1) all exhibit the elevated protein levels of P4Hα1 and VEGF after 48 h culture in hypoxic than normoxic conditions.

FIGS. 7A-7B. (FIG. 7A) Workflow for analysis of Hyp-BK production by hypoxic pancreatic cells. (FIG. 7B) Representative MALDI-TO MS analysis data of nanopore-enriched BK (m/z 1060.62) and Hyp-BK(m/z 1076.62) peptides.

FIGS. 8A-8B. (FIG. 8A) The protein level of P4Hα1 was decreased after transduction with shRNA lentiviral particles. (FIG. 8B) Transduction with P4Hα1 shRNA lentiviral particles led to no significant difference in Hyp-BK/BK ratios between the hypoxic and normoxic cells.

FIGS. 9A-9B. Consistent performance of Hyp-BK/BK. The ratio provides more consistent results compared with either Hyp-BK or BK alone, as multiple cycles of freeze/thaw (FIG. 9A) and prolonged storage (FIG. 9B) of samples would not affect the readout of the Hyp-BK/BK ratio.

### DETAILED DESCRIPTION

Hypoxia is present to some extent in most solid malignant human tumors because of an imbalance between the limited oxygen delivery capacity of the abnormal vasculature and the high oxygen consumption of tumor cells. Numerous observational and therapeutic studies in various tumor types have shown that hypoxia is a key determinant of cancer behavior and treatment outcomes. Although imaging techniques have been used to measure tumor hypoxia, these techniques are not sufficiently accurate to describe tumor hypoxia owing to the rapid, dynamic changes in hypoxic status. It was hypothesized that blood-based biomarkers may have the potential to fill the gaps in current tumor hypoxia profiling strategies. In particular, peptidomics-based analyses will allow the determination of peptide signatures unique to specific hypoxic conditions within tumors because whereby proteolytic activity and post-translational modifications play active roles in the tumor microenvironment.

The present studies confirmed that hypoxia positively regulates hypoxia-inducible factor 1 (HIF-1) and its downstream effector, prolyl 4-hydroxylase subunit alpha-1 (P4HA1) in pancreatic cancer cells. Consequently, one of the major P4HA1 catalytic substrates, hydroxylated bradykinin (hyp-BK), is released from hypoxic tumors into the blood. Using nanotrap-enabled peptide fractionation, it was demonstrated that the hyp-BK/BK ratio can serve as a hypoxia-specific marker and perform consistently in clinical serum samples when quantified by matrixassisted laser desorption-ionization time-of-flight (MALDI-TOF) mass spectrometry. Specifically, it was found that the hyp-BK/BK ratio is significantly elevated in patients with pancreatic cancer who later showed poor responses to anticancer treatment.

The current findings potentially dramatically change the paradigm of personalized therapy for pancreatic cancer, consequently improving the quality of life of patients and their families. Moreover, the biomarker discovery assay is also adaptable to other types of cancer, thus broadening the impact and potential benefits of this research.

In the example below, pancreatic cancer was chosen as the hypoxic tumor model to perform the biomarker discovery study. It was confirmed that hypoxia positively regulates hypoxiainducible factor 1 (HIF-1) and its downstream effector, prolyl 4-hydroxylase subunit alpha-1 (P4HA1) in pancreatic cancer cells. Consequently, one of the major P4HA1 catalytic substrates, hydroxylated bradykinin (hyp-BK) is released from the hypoxic tumor into the blood. The data suggested that the ratio of Hyp-BK to remaining unmodified bradykinin (hyp-BK/BK) in blood may serve a hypoxia-specific marker and could perform consistently in clinical serum samples when quantified using MALDI-TOF MS. The study has demonstrated that the pre-therapy levels of hyp-BK/BK are significantly elevated in patients with pancreatic cancer who later showed poor responses to neoadjuvant treatment.

Certain embodiments of the invention will have a significant impact on biomarker discovery for predicting prognoses. From the clinical perspective, in certain embodiments, the invention identified reliable and accurate biomarkers for tumor hypoxia. The biomarkers are used to predict the outcomes of anticancer treatment, facilitating decision-making in personalized cancer treatment. From a basic research perspective, in certain embodiments, this invention elucidates the correlation between tumor hypoxic status and the blood peptide marker (i.e., the hyp-BK/BK ratio). It is important to note that the hyp-BK/BK ratio is a more accurate, convenient, and consistent marker for predicting treatment response. The clinical implementation of this biomarker has a solid basis in basic mechanistic studies.

The accessibility of measuring blood peptides is exemplified by prompt measurement using a nano-MALDI MS platform that will enable highthroughput, rapid detection of circulating peptide markers for tumor hypoxia. The strategy is noninvasive, fast, and accurate compared with conventional methods for evaluating tumor hypoxia. The method eliminates the necessity of developing specific hypoxia-specific probes for particular imaging studies, which can be time-consuming.

Methods of Detecting

In certain embodiments, the present invention provides a method of detecting hypoxia status in a tumor cell comprising (a) detecting a level of hydroxyprolyl BK (Hyp-BK) peptides in a patient sample, and (b) detecting a level of bradykinin (BK) peptides the patient sample, wherein an increased Hyp-BK/BK ratio as compared to the Hyp-BK/BK ratio in a comparable normal cell is a marker for tumor hypoxia.

In certain embodiments, the tumor is a pancreatic cancer cell.

In certain embodiments, the Hyp-BK/BK ratio is increased by at least 10% over the Hyp-BK/BK ratio in a comparable normal cell. In certain embodiments, the ratio is increased by at least 10%, 20%, 30%, 40%, 50%, 60%, 70%, 80%, 90% 95%, or 99%.

In certain embodiments, the patient sample is a blood sample.

In certain embodiments, the blood sample is a plasma sample.

In certain embodiments, the patient sample is subjected to an enrichment method of nanopore fractionation for circulating peptides prior to the detection steps.

In certain embodiments, the levels of Hyp-BK and BK are determined by Western blot, matrix-assisted laser desorption/ionization time-of-flight mass spectrometry (MALDI-TOF MS).

In certain embodiments, the levels of Hyp-BK and BK in the patient sample is by MADLI-TOF MS.

In certain embodiments, the present invention provides a method of detecting hypoxia status in a tumor cell comprising (a) detecting a level of hydroxyprolyl BK (Hyp-BK) in a patient sample, and (b) detecting a level of bradykinin (BK) the patient sample, wherein BK and Hyp-BK peptides from the patient sample are subjected to nanopore-based fractionation and analyzed by matrix-assisted laser desorption/ionization time-of-flight mass spectrometry (MALDI-TOF MS).

Methods of Treating

In certain embodiments, the present invention provides a method of treating a hyperproliferative disorder in a patient in need thereof comprising administering to a patient identified as having a low plasma hydroxyprolyl BK (Hyp-BK)/protein bradykinin (BK) ratio (as compared to a normal or control level in patient) a pharmaceutical composition comprising a chemotherapeutic agent.

In certain embodiments, the hyperproliferative disorder is a cancer.

In certain embodiments, the cancer is pancreatic cancer.

In certain embodiments, the present invention provides a method of treating a hyperproliferative disorder in a patient in need thereof comprising administering to a patient identified as having a high plasma hydroxyprolyl BK (Hyp-BK)/protein bradykinin (BK) ratio (as compared to a normal or control level in patient)a pharmaceutical composition comprising a chemotherapeutic agent.

In certain embodiments, the hyperproliferative disorder is a cancer.

In certain embodiments, the cancer is pancreatic cancer.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Gastric cancer, as used herein, includes stomach cancer, which can develop in any part of the stomach and may spread throughout the stomach and to other organs; particularly the esophagus, lungs, lymph nodes, and the liver.

A “chemotherapeutic agent” is a biological (large molecule) or chemical (small molecule) compound useful in the treatment of cancer, regardless of mechanism of action. Classes of chemotherapeutic agents include, but are not limited to alkylating agents, antimetabolites, spindle poison plant alkaloids, cytotoxic/antitumor antibiotics, topoisomerase inhibitors, proteins, antibodies, photosensitizers, and kinase inhibitors. Chemotherapeutic agents include compounds used in “targeted therapy” and non-targeted conventional chemotherapy.

The term “mammal” includes, but is not limited to, humans, mice, rats, guinea pigs, monkeys, dogs, cats, horses, cows, pigs, and sheep.

The phrase “pharmaceutically acceptable” indicates that the substance or composition must be compatible chemically and/or toxicologically, with the other ingredients comprising a formulation, and/or the mammal being treated therewith.

Dosages, Formulations and Routes of Administration of the Agents

In certain embodiments, the agents of the invention are administered to result in a reduction in at least one symptom associated with a disease. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems, which are well known to the art.

In certain embodiments, the present invention envisions treating a hyperproliferative disorder (e.g., cancer) in a mammal by the administration of a therapeutic agent. In certain embodiments, the Administration of the therapeutic agents in accordance with the present invention may be continuous or intermittent, depending, for example, upon the recipient's physiological condition, whether the purpose of the administration is therapeutic or prophylactic, and other factors known to skilled practitioners. In certain embodiments, the administration of the agents of the invention may be essentially continuous over a preselected period of time or may be in a series of spaced doses. Both local and systemic administration is contemplated.

In certain embodiments, one or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the tumor. Alternatively, the therapeutic agent may be introduced systemically (e.g., intravenously). In another example, the therapeutic agent may be introduced intramuscularly for viruses that traffic back to affected neurons from muscle, such as AAV, lentivirus and adenovirus. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

In certain embodiments, when the therapeutic agents of the invention are prepared for administration, they are combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules, as a solution, a suspension or an emulsion.

In certain embodiments, pharmaceutical formulations containing the therapeutic agents of the invention can be prepared by procedures known in the art using well-known and readily available ingredients. In certain embodiments, the therapeutic agents of the invention can also be formulated as solutions appropriate for parenteral administration, for instance by intramuscular, subcutaneous or intravenous routes.

In certain embodiments, the pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

In certain embodiments, the pharmaceutical formulations of the present invention may include, as optional ingredients, pharmaceutically acceptable carriers, diluents, solubilizing or emulsifying agents, and salts of the type that are well known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0, saline solutions and water.

The invention will now be illustrated by the following non-limiting Examples.

### EXAMPLE 1

### Hypoxia-Induced Hydroxylated Peptide in Blood as Predictive Biomarker for Prognosis of Pancreatic Cancer Therapy

Abstract

Tumor hypoxia is linked to poor prognosis due to its role in promoting cancer progression and metastatic cascades. Evaluating tumor hypoxia status to predict patient outcomes is clinically challenging due to the current lack of rapid, non-invasive and accurate methods. Hypoxia positively regulates the al subunit of prolyl-4-hydroxylase (P4H) in several tumors, P4Hα1 expression is involved in hypoxia-inducible modification of the plasma protein bradykinin (BK) to hydroxyprolyl BK (Hyp-BK), leading to the hypothesis that hypoxic status could be detected by changes in Hyp-BK. It was found that P4Hα1 expression was selectively increased in malignant vs. non-malignant pancreatic cancer tissues and in accordance with the expression level of tissue Carbonic Anhydrase 9, which is a suggested endogenous marker of tumor hypoxia. Finally, systemic Hyp-BK/BK ratios in pre-treatment plasma samples of pancreatic cancer patients were found to inversely correlate with patients' response to treatment, suggesting that plasma Hyp-BK/BK can be used a non-invasive, surrogate marker for tumor hypoxia and disease prognosis in response to anticancer therapy.

Introduction

Tumor microenvironments are known to regulate carcinogenesis and metastasis, and thus impact responses to cancer therapy. Hypoxia is one of the most significant biochemical and pathophysiological changes that occurs in the tumor microenvironment and is an important clinical feature that is closely linked to poor patient outcomes. Recent studies highlight the importance of tumor hypoxia in altering cell metabolism, promoting cancer stem cell growth and increasing extracellular matrix deposition, remodeling and degradation, which can drive cancer metastasis. Detecting tumor hypoxia status is therefore of great clinical interest; however, the gold standard for assessing tumor hypoxia is highly invasive, as it requires insertion of polarographic electrodes into a tumor mass, and is subject artifacts due to electrode placement and other factors. Less invasive methods, such as electron paramagnetic resonance, ¹⁹F-magnetic resonance spectroscopy, overhauser-enhanced MRI, PET imaging and others are not suitable for routine clinical use due to regulatory restraints, safety concerns and/or their uncertain correlations with prognosis.

Non-invasive biomarker approaches to measure tumor hypoxia are thus highly desirable, and circulating biomarkers modified by tumor prolyl-4-hydroxylase (P4H) activity represent attractive candidates. Tumor hypoxia increases hypoxia inducible factorla (HIF-1α) to upregulate P4Hα1 expression and P4H activity, which is essential for normal collagen folding and stability, including during tumor remodeling but which also modifies BK, a plasma peptide involved in blood pressure regulation and inflammation, to form Hyp-BK. Therefore, it was hypothesized that plasma Hyp-BK levels could be used as a predictive marker for pancreatic cancer treatment outcomes. This report describes a rapid, high-throughput method for monitoring plasma Hyp-BK/BK ratios as a surrogate marker for tumor hypoxia and a predictive marker for treatment response.

Materials and Methods

Cell Lines and Culture

Pancreatic cancer cell lines (AsPC-1, BxPC-3, Colo357, MIA PaCa-2, and PANC-1), normal pancreas cell lines (HPDE and HPNE), and normal pancreas cell lines overexpressing a G12V K-Ras mutant allele (HPDEK and HPNEK) were kindly provided by Professor Paul Chiao (MD Anderson Cancer Center, Houston Tex.). AsPC-1 and BxPC-3 cells were maintained in RPMI-1640 (HyClone, GE Healthcare Life Sciences) supplemented with 10% fetal bovine serum (Gibco, Thermo Fisher Scientific). Colo357, MIA PaCa-2, and PANC-1 cells were grown in DMEM with 10% fetal bovine serum. HPDE and HPDEK cells were cultured in Keratinocyte-SFM medium (17005-042, Life Technologies). HPNE and HPNEK cells were grown in 75% DMEM, 25% M3: Base F Medium (INCELL), and 5% fetal bovine serum. Cells were cultured under at 37° C. in a humidified incubator under normoxic conditions (5% CO₂, 95% air (20% O₂)) unless stated otherwise. Cells cultured under hypoxic conditions were placed in an incubator chamber (Stemcell) that was purged with a mix of 5% CO₂, 94% N₂ and 1% O₂ at a rate of 20 liters/min for 10 min, and then sealed and incubated in a conventional CO₂ incubator.

Tissue Array

One human pancreatic cancer tissue array was purchased from BIOMAX (OD-CT-DgPan01-006). Other tissue arrays were kindly provided by Professor Haiyong Han (Translational Genomics Research Institute, Phoenix Ariz.). Immunohistochemistry staining was performed as previously described using a rabbit anti-P4Hα1 antibody (84398, Novus, 1:500) or a rabbit anti-CA9 antibody (417, Novus, 1:100).

Western Blot

Whole cell lysates prepared using M-Per Mammalian Protein Extraction Reagent (78501, Thermo Scientific), nuclear and cytoplasmic proteins were extracted with NE-PER™ Nuclear and Cytoplasmic Extraction Kit (78833,Thermo Scientific), then proteins were fractionated by SDS-PAGE on 4-15% Mini-Protean TGX Precast Protein Gels (Bio-Rad), transferred to nitrocellulose membranes using a Trans-Blot Turbo system (Bio-Rad), and hybridized with mouse anti-HIF-1α (113642, Abcam, 1:500), rabbit anti-P4Hα1 (84398, Novus, 1:500), rabbit anti-GAPDH (14C10, Cell Signaling Technology, 1:1000) VEGF antibodies and HRP-conjugated rabbit anti-IgG secondary antibody using standard methods. Immunoreactive proteins were visualized using ChemiDoc XRS+ imaging system (Bio-Rad), and optical density values were normalized to GAPDH loading controls for relative quantification.

Real-Time Reverse Transcription Quantitative PCR (RT-qPCR)

Total RNA extracts were generated from cultured cells using TRIzol reagent (Thermo Fisher Scientific) and processed using an iScript Reverse Transcription Supermix (Biorad) to prepare cDNA for triplicate quantitative PCR assays using iTaq Universal SYBR Green Supermix (Bio-Rad). Target mRNA expression was normalized to a GAPDH internal control and expressed as a 2-Δ(ΔCt) value of the hypoxic vs. normoxic target gene expression. PCR products were identified by agarose gel electrophoresis and direct sequencing. PCR primers are listed in Table 1.

Chromatin Immunoprecipitation (ChIP)-PCR

ChIP assays were performed using EZ-Magna ChIP Kits (17408, EMD Milipore), Colo357 cells cultured in normoxic or hypoxic environments for 24 h and a mouse anti-HIF-1α antibody (ab1, Abcam). ChIP DNA fractions were analyzed by qPCR and normalized to IgG-pulled down DNA using primers listed in Table 1. Each ChIP assay was performed in triplicate.

TABLE 1
The primer sequences for RT-qPCR and ChIP-PCR

| Primer | Assay | Forward 5′-3′ | Reverse 5′-3′ |
|---|---|---|---|
| Actin | RT- | AGAGCTACGAGCTGCCTGAC | AGCACTGTGTTGGCGTACAG |
| | qPCR | | |
| HIF-1α | RT- | AGATCTCGGCGAAGCAAAGA | CGGCATCCAGAAGTTTTCTCACAC |
| | qPCR | GT | |
| P4Hα1 | RT- | AGGACATGTCGGATGGCTTC | TCTTGCAGCCGAAACAGAGCTT |
| | qPCR | ATCT | |
| P4Hα1 | ChIP- | CTCTCGGCCTCAGACTCC | AGGGAGGAGGCGACTGCA |
| promoter | PCR | | |

Immunoprecipitation and In Vitro P4Hα1 Activity Assay

Cell lysate protein was quantified by BCA protein assay kits (Pierce) and equal amounts of total protein were immunoprecipitated with protein A agarose (Pierce) and rabbit anti-P4Hα1 antibody (84398, Novus), rabbit anti-Bradykinin antibody (Biotin) or rabbit IgG (Biotin) Isotype Control respectively, using western blot analyses to evaluate pulldown efficiency or P4Hα1 expression level. P4Hα1 Enzymatic activity was assayed using the P4Hα1-enriched fraction and 0.1 mM BK in a 1 mL reaction volume containing 50 μM of Tris-HCl (pH 7.8), 0.05 μM FeSO4, 0.1 μM α-ketoglutarate, 1 μM ascorbate, 0.1 mg catalase, 0.1 μM dithiothreitol and 2 mg/ml bovine serum albumin, which was incubated at 37° C. for 1 h. BK and Hyp-BK peptides were subjected to nanopore-based fractionation and then analyzed by matrix-assisted laser desorption/ionization time-of-flight mass spectrometry (MALDI-TOF MS) using a 4700 proteomics analyzer (Applied Biosystems) to calculate Hyp-BK/BK ratios.

Nanoporous Fractionation

Nanoporous silica chips were fabricated and 5 μL of a protein sample was dispensed into duplicate wells, incubated for 30 min at 25° C. in a humidified chamber, decanted to remove unbound protein. Sample wells were then washed 4× with 10 μl of deionized water, after which peptides were extracted by applying 6 μl of elution buffer (50% acetonitrile (ACN)/0.1% trifluoroacetic acid (TFA)) to each well and incubating the chip for 90 min at 25° C. in a humidified chamber, after which aspirated supernatants were subjected to MALDI-TOF MS analysis.

MALDI-TOF MS

After nanopore fractionation, 1.5 μl of each eluate was directly spotted onto the target plate and allowed to dry at room temperature then spotted with 1.5 μl of matrix solution (4 g/L of a-cyano-4-hydroxycinnamic acid in 50% ACN and 0.1% TFA). MALDI-TOF spectra were generated using a 4700 proteomics analyzer with a fixed laser intensity of 5500 and a positive reflector mode in the 900-2000 Da range with a focus of 1000 Da. Mass spectra for each sample were acquired from 3000 total shots (50 shots for 60 sub-spectra). MS spectra were calibrated externally and raw data were processed using FLEX software v. 1.2.1 (AB SCIEX).

shRNA and Transduction

Colo357 cells were transduced with P4Hα1 shRNA lentiviral particles (Santa Cruz), which was later detected by Western blot to confirm the knockdown efficiency.

Collection of Blood Samples

Pre-treatment blood samples and subsequent prognostic information were obtained from 34 pancreatic cancer patients at the MD Anderson Cancer Center and 40 pancreatic cancer patients at Translational Genomics Research Institute who had given prior written informed consent for study participation, and this material was used in accordance with all relevant institutional and federal guidelines and regulations. Patient responses to therapeutic interventions were evaluated by pathologists, using the percentage of viable malignant cells present in tumor biopsies post-therapy to divide patients into good vs. poor response groups (<10% viable cells vs. ≥10% viable tumor cells, respectively).

Statistics

Data were analyzed using GraphPad Prism software. Student's two-tailed t-test was performed for paired samples. Correlation analysis between treatment response and Hyp-BK/BK was performed by Spearman Rank Correlation. All data were expressed as mean±SD unless stated otherwise.

Results

HIF-1α Positively Regulates P4Hα1 Under Hypoxia

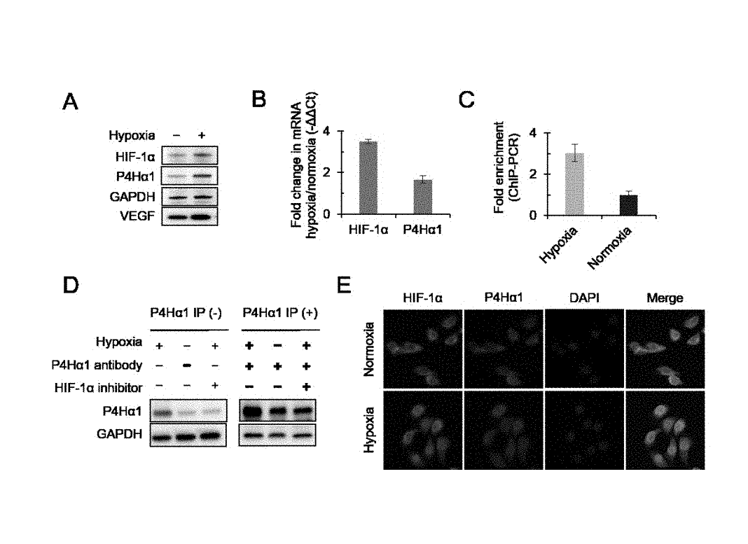

Elevated Tumor HIF-1 expression predicts poor pancreatic cancer prognosis, but HIF-1 has a very short half-life (˜5 min) and its measurement in highly invasive tumor biopsies is thus non-feasible in standard practice. HIF-1α increases during hypoxia in breast cancer and prostate cancer are also reported to upregulate P4Hα1 expression, which could also increase P4H activity and the post-translational modification of specific plasma proteins. To determine if hypoxia also induces P4Hα1 in pancreatic cancer, the pancreatic cancer cell line Colo357 was cultured for 48 h under normoxic or hypoxic conditions (20% vs.1% 02), finding that P4Hα1 and hypoxia-induced VEGF protein levels were elevated after 48 h culture in hypoxia (FIG. 1A). P4Hα1 protein level increased accordingly with the hypoxic culture time prolongation (FIG. 6A), corresponding to HIF-1α and P4Hα1 mRNA increases (FIG. 1B). ChIP-PCR analysis also detected a ˜3-fold increase in HIF-1α binding to the P4Hα1 promoter in hypoxic vs. normoxic Colo357 cells (FIG. 1C), suggesting that HIF-1α directly promotes pancreatic P4Hα1 expression during hypoxia, similar to its previously observed regulation in other physiological and pathological processes, including breast cancer metastasis and normal bone development. After hypoxia treatment, Colo-357 cells showed different HIF-1α sub-cellular localisations compared within either nuclear fraction or cytoplasmic fraction under normoxia conditions. The localisation of P4Hα1 also showed similar change following hypoxic treatment (FIG. 6B). The differential responses to hypoxia were evaluated among several human pancreatic cancer cell lines (i.e., AsPC-1, BxPC-3, MIA PaCa-2, and PANC-1). Consistently, the protein levels of HIF-1α, P4Hα1, and hypoxia-induced VEGF were increased under hypoxia for all the cell lines tested (FIG. 6C). Hypoxia significantly increased P4Hα1 protein expression, which was fully attenuated upon treatment with the specific HIF-1α inhibitor digoxin. This pattern was retained after enriching for P4Hα1 expression by immunoprecipitation (FIG. 1D). Supporting this notion, immunofluorescence microscopy also shows upregulation of HIF-1α and P4Hα1 in hypoxic cells, and their subcellular localization is consistent with previous report (FIG. 1E).

Hypoxia-Induced P4Hα1 Promotes Hyp-BK/BK in Pancreatic Cancer Cells

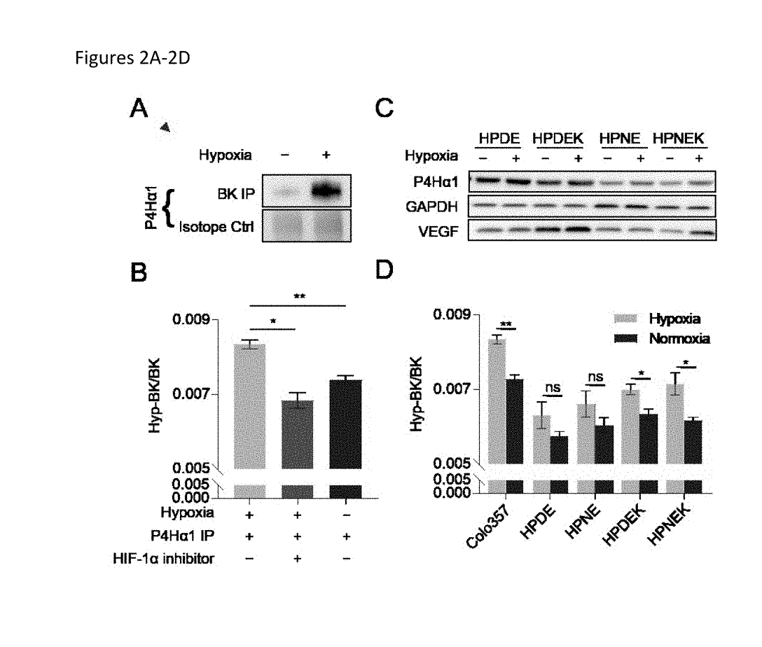

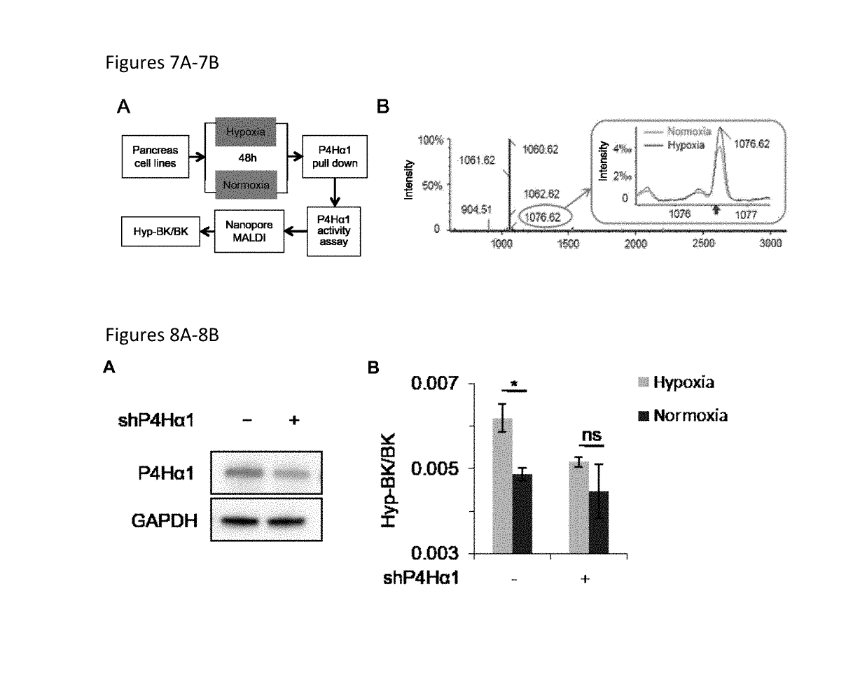

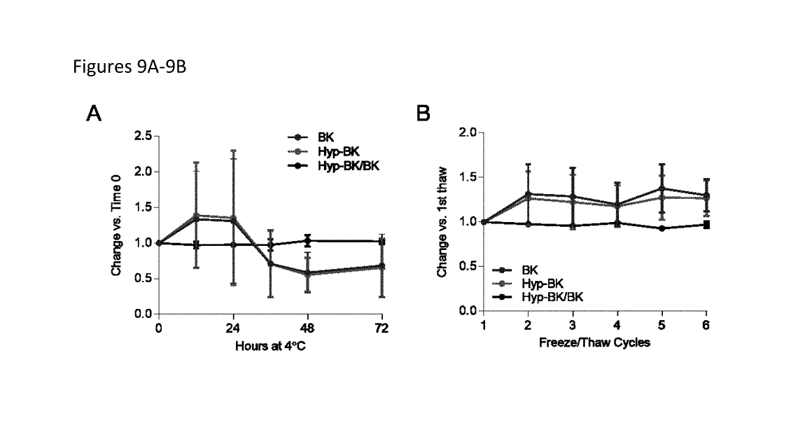

To confirm that hypoxia-induced P4Hα1 directly determines the level of Hyp-BK/BK, an in vitro assay was adapted to quantify P4Hα1-mediated Hyp-BK synthesis. BK was immunoprecipitated from cell lysates cultured under normoxic and hypoxic conditions, increased P4Hα1 expression in hypoxic BK pulled down sample was observed (FIG. 2A). P4Hα1 was immunoprecipitated from cell lysates of Colo357 cell cultured under normoxic and hypoxic conditions and quantified Hyp-BK synthesis as the ratio of nanopore-fractionated BK and Hyp-BK peptides detected by mass spectrometry (FIG. 7A). Hypoxia significantly increased P4Hα1 protein expression was reflected in hypoxia and HIF-1α-dependent increases in Hyp-BK/BK ratios produced by P4Hα1-enriched samples (FIG. 2B), which were detected by a 16 Da mass to charge (m/z) shift marking the hydroxyl modification that distinguishes Hyp-BK from BK (FIG. 7B: m/z 1076.62 vs. 1060.62, respectively). The Hyp-BK/BK ratio was found to provide more consistent results than either Hyp-BK or BK alone when samples were subjected to extended incubation times at 4° C. or repeated freeze/thaw cycles (FIGS. 9A and B). Additionally, when P4Hα1 was knocked down in Colo357 by shRNA, even the efficiency was not there was no significant difference in Hyp-BK/BK ratios between the hypoxic and normoxic cells, further highlighting the critical role of P4Hα1 (FIGS. 8A-8B). Taken together, these data suggest that the level of Hyp-BK/BK is directly determined by the hypoxia-induced P4Hα1 activity and positively correlated with hypoxia. Therefore, Hyp-BK/BK can be a marker to reflect the hypoxic status in pancreatic cancer.

Pancreatic Tumor Cells are More Responsive to Hypoxia than Normal Cells

The current data support the notion that Hyp-BK/BK is indicative of cell hypoxic status. Thus, it was tested if Hyp-BK/BK will be significantly elevated during fast tumorous proliferation where the hypoxic microenvironment becomes exacerbated. The differential responses to hypoxia were evaluated among two normal pancreas cell lines (i.e., HPDE and HPNE), and two KRAS-mutant-transformed normal cell lines (i.e., HPDEK and HPNEK). Cancer cells appeared to be more responsive to hypoxia as differential change of P4Hα1 and VEGF (i.e., hypoxia vs. normoxia) was more obvious in cancer cells than the normal ones (FIG. 2C). Indeed, the ratio of Hyp-BK/BK was significantly elevated in the hypoxic tumor cells than the normoxic ones: in Colo357 cells, hypoxia had induced significantly higher level of Hyp-BK/BK. In contrast, the normal cell lines (HPDE or HPNE) failed to demonstrate any significant difference in this ratio between hypoxia and normoxia. Interestingly, two KRAS-mutant-transformed normal cell lines, HPDEK and HPNEK, also showed significantly higher ratio of Hyp-BK/BK for hypoxia (FIG. 2D).

The Expression of P4Hα1 is Correlated with Patient Survival

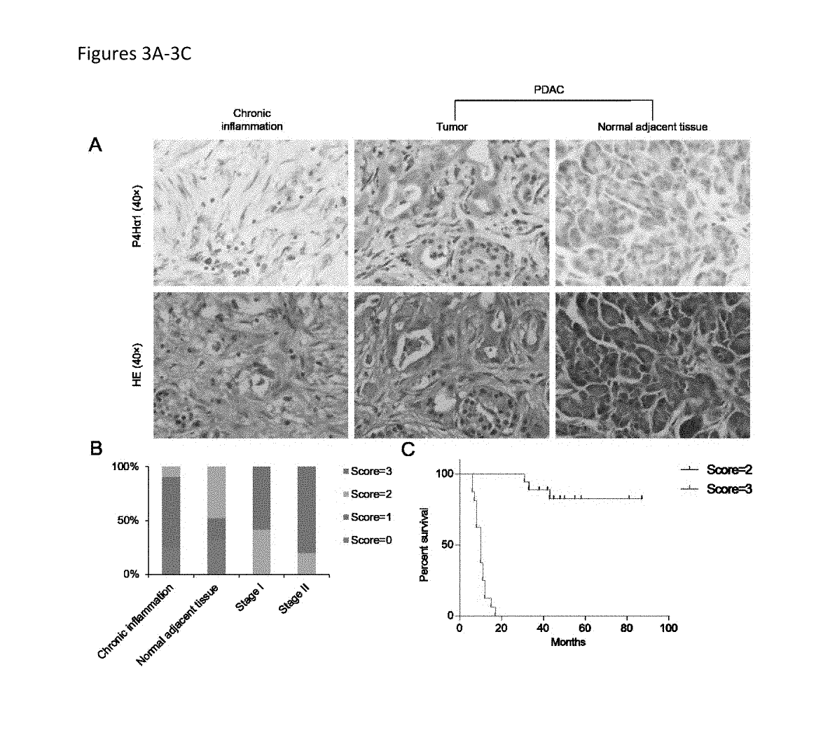

P4Hα1 expression level has been reported to predict disease progression or prognosis in prostate cancer and breast cancer. Therefore, P4Hα1 expression was analyzed in a pancreatic tissue array derived from patients with and without pancreatic cancer, including patients with pancreatic ductal adenocarcinoma with (4 cases) and without (77 cases) nerve invasion, ductal hyperplasia (6 cases), chronic inflammation (32 cases) as well as normal adjacent tissue (NAT) from patients with (2 cases) and without (41 cases) tissue degeneration. Strong P4Hα1 staining was detected in pancreatic ductal adenocarcinoma and metastatic tissue samples (FIG. 3A-B), which progressively increased with tumor stage (p<0.05), and was negatively associated with patient survival in the 36 cases with this information (FIG. 3C).

A Nanopore Peptidomic Enrichment Approach to Quantify Blood Hyp-BK/BK Levels

P4Hα1 catalyzes the proline residues in BK to form the hydroxylated BK (Hyp-BK). Because of the low abundance of small molecular weight peptides in human body fluids, direct quantification of these peptides in the blood meets technical challenges on the MALDI-TOF MS detection platform. A nanopore fractionation and enrichment protocol was developed that allows peptides of small molecular weights to be effectively “trapped” and enriched in a nanoporous silica (NPS) chip by excluding abundant high molecular weight proteins. Without interference from overwhelmingly large amounts of serum proteins (e.g. albumins) masking the target peptidomic signals, desirable small peptides, such as BK and Hyp-BK, are readily detected and identified by MALDI MS (FIG. 7B). Accordingly, quantification of BK and Hyp-BK in the clinical blood samples can be achieved by measuring the intensities of the MS peaks.

The Expression of P4Hα1 Indicates Tumor Hypoxic Condition

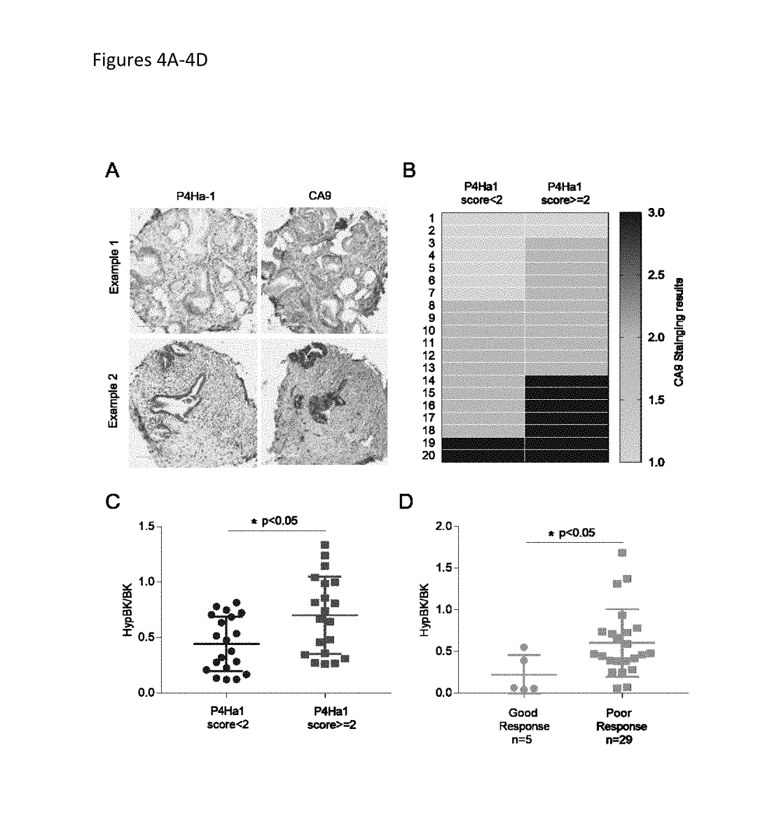

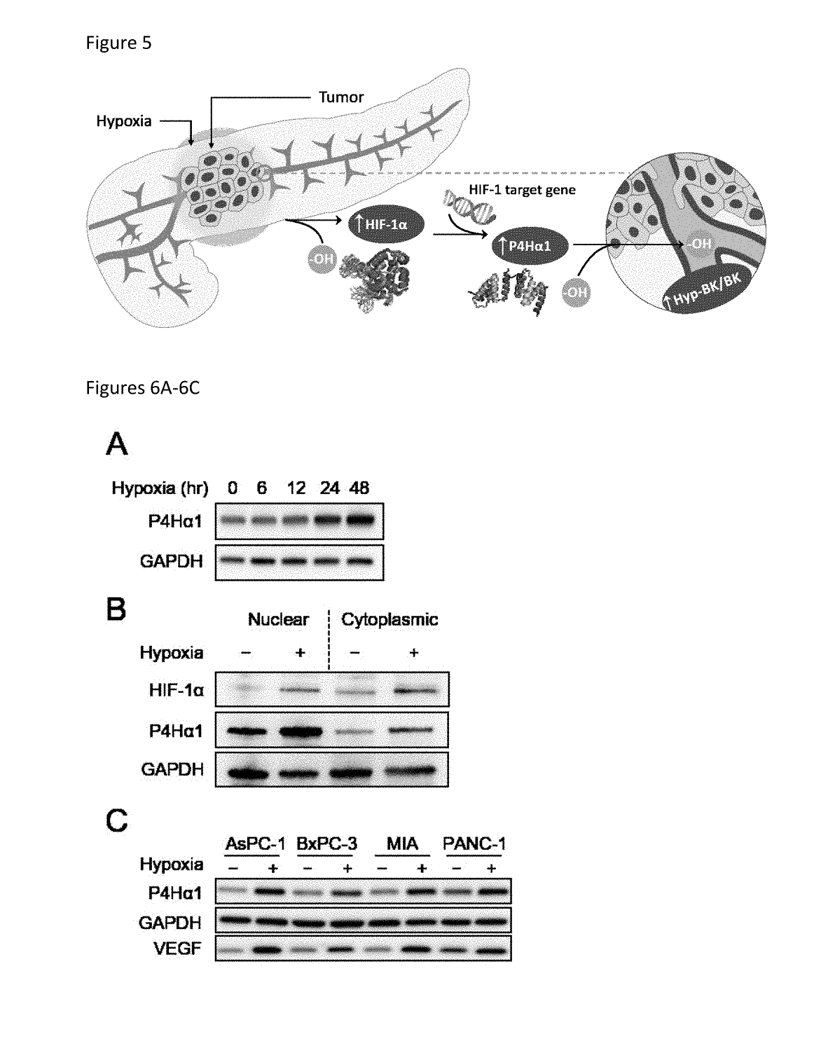

As a transmembrane protein overexpressed in a wide variety of tumor types, CA9 has frequently been associated to hypoxia in the literature. To demonstrate the clinical evaluation of tumor hypoxia by measuring P4Hα1 expression level, it was chosen to compare CA9 immunostaining with P4Hα1 staining in formalin-fixed sections from tumors of 40 pancreatic cancer patients. In addition, Hyp-BK/BK in the pre-treatment serum samples of these patients were measured and analyzed. Among those patients who showed higher tissue P4Hα1 expression level, the tissue CA9 level and serum Hyp-BK/BK ratios were both at higher level (FIG. 4A-C), suggesting that these patients experienced exacerbated tumor hypoxic status. The correlation between patients' P4Hα1 level and age, sex, race, or diabetes history was then analyzed, and no statistical correlation was found.

In Pre-Treatment Plasma Samples the Ratio of Hyp-BK/BK is Correlated with Clinical Prognosis of Anticancer Therapy

To demonstrate the clinical evaluation of tumor hypoxia by measuring the blood Hyp-BK/BK, clinical blood samples were collected from 34 pancreatic cancer patients who later participated in chemo/radiotherapies. The ratios of Hyp-BK/BK in these pre-treatment blood samples were measured and recorded. After therapy, the treatment responses were categorized into two major classes: the “good” response group refers to those who had only <10% viable tumor cells by pathological examination; and the “poor” response group who had >=10% of viable tumor cells. Indeed, such an inverse correlation between the Hyp-BK/BK ratio and the treatment response is significant (p=0.0484 by Mann-Whitney test, p=0.047 by Spearman Correlation), suggesting that the ratio in pre-treatment blood could be used to predict clinical treatment prognosis (FIG. 4C).

The correlation between patients' response and age, sex, race, or diabetes history was then analyzed. No statistical correlation was found except age and response. As age was correlated with patients' response to anticancer therapy (Spearman, p=0.008), the correlation between age and ratio was then analyzed, but no statistical correlation was found between age and ratio (Spearman, p=0.633). In order to eliminate the effect of age on patients' response, ratios in good and poor response groups were compared in the same age span. The age in good response group ranged from 41 to 64 (n=5). Thus, patients less than 40 or more than 65 were removed from the group of poor response (No.4,6,8,9,10,13,15,21,22,23,25,28,30). The ratio in good response group (median (min, max); 0.6584 (0.0466,0.5522); n=5) was significantly lower than poor response (median(min, max); 0.4334(0.0715,1.6853); n=16) (Mann-Whitney, p=0.048), and the ratio was statistical correlated with patients' response (Spearman, p=0.044).

In summary, among those patients who later showed effective response to cancer treatment, the Hyp-BK/BK ratios were at lower level in pre-treatment blood samples, suggesting that the tumors might have been less hypoxic when treatment commenced, and therefore more responsive to anticancer therapy. Reversely, higher levels of Hyp-BK/BK were indicative of exacerbated tumor hypoxic status, which may have conferred the poor responses to anticancer therapy.

Discussion

Hypoxia is one of the major features of tumor microenvironment in pancreatic cancer patients. A previous study has found that the average oxygenation was significantly higher in adjacent normal pancreas than tumor tissues in pancreatic ductal adenocarcinoma (PDAC) by polargraphic electrode measurement. However, the oxygen probe is highly invasive because of the intraoperative procedures, making clinical routine difficult. The readout of oxygen will be incorrect when halogenated anesthetics are administered or necrotic tissues are detected. Thus, directly measuring oxygen levels in tumor tissue is not feasible for wide clinical use. Alternatively, other researchers have used endogenous, hypoxia-responsive molecules and their downstream effectors to assess tumor hypoxia indirectly. For instance, numerous lines of evidence indicate that elevated HIF-1 level predicts worse prognosis in patients with pancreatic cancer. However, HIF-1α accumulates rapidly in transcriptional response to hypoxia but has a very short half-life of approximately 5 min, making it a real challenge for sample collection in clinical routine. Thus, a reliable, sensitive, noninvasive, and high-throughput strategy is in urgent need for hypoxia assessment and prognostic prediction.

Hypoxia is linked to a poor outcome in cancer patients and may be used to predict the progression-free and overall survival rates. Admittedly, poor prognosis can be ascribed to complex factors. However, hypoxia is one of the most important aspects due to its multidimensional biological effects. Particularly, hypoxia promotes extracellular matrix (ECM) re-modelling to facilitate cancer metastasis, where the P4Hα1 activity is critical. Elevated mRNA and protein levels of P4Hα1 by hypoxic induction have been observed for multiple cancers during metastasis, including breast and prostate cancers. Others found that P4Hα1 mRNA and protein levels were highly increased under hypoxia in breast cancer cells MDA-MB-231, and inhibition or knockdown of HIF-1α blocks hypoxia-induced P4Hα1 expression, which is consistent with the hypothesis, HIF-1α upregulates P4Hα1 under hypoxia, in pancreatic cancer cells. Therefore, in the current study, the regulatory mechanism of hypoxia on P4Hα1 and its catalytic products, Hyp-BK was interrogated. It was found that hypoxia-induced P4Hα1 activity accounted for the increased Hyp-BK/BK ratio: firstly, under hypoxia, both HIF-1α and P4Hα1 are significantly upregulated at transcriptional and translational levels; secondly, the ChIP data indicate that P4Hα1 is subject to direct regulation by HIF-1α; thirdly, and most importantl, the in vitro P4Hα1 activity assay provides direct evidence that Hyp-BK and BK are subject to regulation of hypoxia through the HIF-1α-P4Hα1 pathway, as the pharmacological inhibitor of HIF-1α (i.e., digoxin) abolished elevation of Hyp-BK/BK under hypoxia; lastly, Hyp-BK/BK may be specific for hypoxic tumors, since generally only pancreatic cancer cell lines displayed significantly higher levels of Hyp-BK/BK under hypoxia compared with the normal pancreas cell lines. Therefore, the ratio of Hyp-BK/BK can reliably reflect the tumor hypoxic status.

This study provides the molecular and clinical evidence that the Hyp-BK/BK ratio directly reflects the hypoxic status. Previous work has demonstrated that circulating peptides in the blood are usually valuable because they can be important metabolic/catalytic products of enzymes that are implicated or regulated by pathophysiological processes like cancer. Therefore, it is conceivable that in pancreatic cancer, the levels of Hyp-BK and BK may be predetermined by the hypoxic microenvironment in tumor through the HIF-1α-P4Hα1 signaling pathway. The Hyp-BK and BK peptides are then released into the blood and it is proposed that the Hyp-BK/BK ratio in blood can be measured to determine the tumor hypoxic status, which may serve as an effective predictive marker for anticancer treatment response among the patients. Significantly, combining the effective enrichment method of “nanopore” fractionation for circulating peptides and the sensitive detection method of MADLI-MS overcomes the technical challenges and limitations imposed by conventional methods and developed a noninvasive, consistent, and high-throughput platform to detect, quantify, and evaluate tumor hypoxia using pancreatic cancer as the disease model.

Circulating small molecular weight peptides are promising biomarker candidates for human diseases. Due to their low abundance in the human fluids, consistent, reliable, and accurate quantification is challenging. The detecting platform addresses the critical problem of enrichment by trapping the target peptides in a nanoporous silica chip based on size exclusion where the interference from abundant, high molecular weight proteins is significantly minimized. Moreover, depending on the properties of target peptides, the size and physiochemical properties of the nanopore can be further modified to accommodate particular needs of different studies. Therefore, the method is a major advancement for biomarker discovery studies. Being convenient and effective, it can be easily adapted to high-throughput formats.

It is worth pointing out that the marker of Hyp-BK/BK ratio may be tumor-specific due to the following observations: (1) only pancreatic cancer cell lines displayed high levels of Hyp-BK/BK ratio when subjected to hypoxic culturing condition; (2) the KRAS-mutant-transformed normal pancreas cell lines (i.e., HPDEK and HPNEK) also showed the same pattern. Since this type of K-RAS mutant is frequently found in aggressive and malignant cancers, it is proposed that the mutant K-RAS may positively modulate P4Hα1 functionality under hypoxia and therefore promote the malignant transformation of a normal cell, resulting in elevated Hyp-BK/BK ratios. Following the logic, it is possible that during the normal-to-oncogenic transition, response to hypoxia becomes tumor-specific.

In summary, the results indicate that elevated P4Hα1 expression 1) distinguishes malignant from non-malignant pancreatic tissue, 2) is required for hypoxia-inducible Hyp-BK production by prolyl hydroxylase activity in pancreatic cancer cells, and 3) is increased by transformation of normal pancreas cells with a K-RAS mutation found in early tumors and associated with aggressive tumor prognoses, while 4) systemic Hyp-BK/BK ratios in pre-treatment plasma samples of pancreatic cancer patients were found to inversely correlate with treatment outcomes. Rapid, non-invasive and accurate detection of tumor hypoxic stress or analysis of a biomarker predicting treatment response can be used to guide individualized therapy to optimize treatment plans. The study offers a unique, plasma peptide detection strategy for assessing tumor hypoxia and predicting the response to standard regimens.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of detecting hypoxia status in a tumor cell comprising
(a) detecting a level of hydroxyprolyl BK (Hyp-BK) peptides in a patient sample, and
(b) detecting a level of bradykinin (BK) peptides the patient sample,
wherein an increased Hyp-BK/BK ratio as compared to the Hyp-BK/BK ratio in a comparable normal cell is a marker for tumor hypoxia.

2. The method of claim 1, wherein the tumor is a pancreatic cancer cell.

3. The method of claim 1, wherein the Hyp-BK/BK ratio is increased by at least 10% over the Hyp-BK/BK ratio in a comparable normal cell.

4. The method of claim 1, wherein the patient sample is a blood sample.

5. The method of claim 4, wherein the blood sample is a plasma sample.

6. The method of claim 1, wherein the patient sample is subjected to an enrichment method of nanopore fractionation for circulating peptides prior to the detection steps.

7. The method of claim 1, wherein the levels of Hyp-BK and BK are determined by Western blot, matrix-assisted laser desorption/ionization time-of-flight mass spectrometry (MALDI-TOF MS).

8. The method of claim 7, wherein the levels of Hyp-BK and BK in the patient sample are measured by MADLI-TOF MS.

9. A method of detecting hypoxia status in a tumor cell comprising
(a) detecting a level of hydroxyprolyl BK (Hyp-BK) in a patient sample, and
(b) detecting a level of bradykinin (BK) the patient sample,
wherein BK and Hyp-BK peptides from the patient sample are subjected to nanopore-based fractionation and analyzed by matrix-assisted laser desorption/ionization time-of-flight mass spectrometry (MALDI-TOF MS).

10. A method of treating a hyperproliferative disorder in a patient in need thereof comprising administering to a patient identified as having a low or high plasma hydroxyprolyl BK (Hyp-BK)/protein bradykinin (BK) ratio as compared to a normal or control value a pharmaceutical composition comprising a chemotherapeutic agent.

11. The method of claim 10, wherein the patient has a low Hyp-BK/BK ratio as compared to the normal or control value.

12. The method of claim 11, wherein the hyperproliferative disorder is a cancer.

13. The method of claim 12, wherein the cancer is pancreatic cancer.

14. The method of claim 10, wherein the patient has a high Hyp-BK/BK ratio as compared to the normal or control value.

15. The method of claim 14, wherein the hyperproliferative disorder is a cancer.

16. The method of claim 15, wherein the cancer is pancreatic cancer.
